(19) United States
(12) Patent Application Publication
(10) Pub. No.: US 2026/0045370 A1
(43) Pub. Date: Feb. 12, 2026

(54) System and Method for Tracing Lot Codes of Food Products in Supply Chain

(71) Applicant: iFoodDecisionSciences, Inc. (IFDS), Seattle, WA (US)

(72) Inventors: Claire Zoellner, Ithaca, NY (US); Jiyeon Baek, Seattle, WA (US); Andrew W. Kennedy, Durham, NC (US); Charles Robison, Newburgh, NY (US)

(21) Appl. No.: 18/795,850

(22) Filed: Aug. 6, 2024

(51) Int. Cl. G16H 50/80; G06Q 10/087

(52) U.S. Cl. CPC G16H 50/80; G06Q 10/087

(57) ABSTRACT

Systems, apparatuses, and methods that provide information relevant to trace a food or other product back to its source of production to protect the health of consumers during an outbreak of foodborne illness or to optimize supply chains for product freshness. Embodiments provide a way to better communicate traceability information to the final step of the present food chain (i.e., retail or foodservice outlet) than conventional approaches, which provide little, if any, capability to satisfy the requirements of the FDA and FSMA regulations.

### BACKGROUND

Monitoring the supply and distribution of food products is both complex and important to protecting consumers, in part because of the many aspects that can lead to contamination or can result in illness or disease. The production of a raw food ingredient (such as vegetables or fruit), the processing of an ingredient, the combining of ingredients into a food product, and the storage, distribution and sale of ingredients or food products can each present sources of contamination that may lead to illness, disease, or death. For that reason, the ability to identify and locate points in the supply chain(s) that are the source of a problem is an important feature in efforts to control the spread of potentially harmful raw ingredients or food products. This often requires identifying the problematic “lot” of an ingredient or food product and tracing it back to its source or other location where a problem was first introduced.

The Food and Drug Administration (FDA) is one of the governmental entities responsible for ensuring the safety of food products. As part of its efforts to protect consumers, the FDA was provided with certain regulatory powers under the Food Safety Modernization Act (FSMA). The FSMA gave the FDA authority to regulate the way foods are grown, harvested, and processed.

As part of the FSMA, the FDA issued what is known as the FSMA 204 Food Traceability Rule, or CFR Title 21 Chapter 1 Subchapter A Part 1 Subpart S, a regulation requiring companies that manufacture, process, pack, or hold foods listed on the Food Traceability List (FTL) to capture and share certain key data elements at critical tracking events (CTEs) along the supply chain. This is intended to facilitate more rapid traceback investigations in the event of an outbreak of foodborne illness. Food companies have been sharing basic traceability information across the supply chain, but the new regulation introduces two new variables that must be included for every item and transaction: the Traceability Lot Code (TLC) and the TLC Source.

The TLC is a descriptor, often alphanumeric, and is used to uniquely identify a batch of a food product, where the batch may be expressed as a “lot” or group of “cases” (as examples). The location at which the TLC was originated or created is described by the TLC Source and may include a unique facility registration or identification number, a physical address, or a web address, as examples. Every item (such as an ingredient) for a food on the Food Traceability List FTL must be associated with a TLC and TLC Source. For generalization in this disclosure, we refer to TLC and TLC Source as batch lot and lot source, respectively.

Due to inconsistent recording of batch lot and lot source for individual items and limitations in the ability of warehouse and inventory management systems (WMS, IMS) to capture these per item, this regulatory requirement poses a significant barrier to compliance for warehouses and retailers who make outbound shipments of individual items to final consumers (“the final mile”). This is at least partly because they are unable to provide the batch lot and lot source for every item in a sales transaction to a restaurant, retail store, or institution, etc. Assuming suppliers apply batch lot barcodes to cases of product shipped to a distribution center, the distributor can minimally track batch lots entering their warehouse on a given date and time, but do not have infrastructure or personnel to track when a batch lot leaves the warehouse and where it went. This complicates and, in some cases, may prevent efforts to trace back sources of contamination, illness, or disease to a food product distributed by such a warehouse.

Embodiments are directed to solving these and other disadvantages of conventional approaches to tracing sources of food products, either alone or in combination.

### SUMMARY

The terms “invention,” “the invention,” “this invention,” “the present invention,” “the present disclosure,” or “the disclosure” as used herein are intended to refer broadly to all the subject matter disclosed in this document, the drawings or figures, and to the claims. Statements containing these terms do not limit the subject matter disclosed or the meaning or scope of the claims. Embodiments covered by this disclosure are defined by the claims and not by this summary. This summary is a high-level overview of various aspects of the disclosure and introduces some of the concepts that are further described in the Detailed Description section below. This summary is not intended to identify key, essential or required features of the claimed subject matter, nor is it intended to be used in isolation to determine the scope of the claimed subject matter. The subject matter should be understood by reference to appropriate portions of the entire specification, to any or all figures or drawings, and to each claim.

Embodiments are directed to systems, methods, and apparatuses that provide a way to trace a food product to a source of contamination, illness, or disease as part of protecting the health of consumers. Embodiments provide a way to better monitor the final step of the present food chain (i.e., the distribution or sale of a food product or ingredient to an end user and ultimately a consumer) than conventional approaches, which provide little, if any, capability to satisfy the requirements of the FDA 204 requirements for additional traceability records for certain foods or food items.

Food and food ingredient distributors and retailers seek a relatively low-cost approach to providing more granular traceability information for the final step of the food supply chain. Embodiments of the disclosure are based on treating the problem of traceability as a data problem that can be solved to a sufficient degree by using simulation models and probability theory to calculate the batch lot(s) associated with cases selected for outbound order fulfillment. In this approach, an overall solution relies on traceability information recorded upon receiving items into a distribution center and combines that information with warehouse management system (WMS) or inventory management system (IMS) inventory and transaction data and application of the disclosed algorithm(s) to produce an estimate of the batch lot(s) (i.e., the lot identifiers) leaving the distribution center or retailer in each outbound order.

One output of an embodiment of the proposed solution is an estimate of the likelihood or probability that a specific batch lot (and lot source) was contained in an order shipped from a distribution warehouse. In many situations, this is not only more accurate information than available from conventional approaches but may be sufficient to narrow down the source of a problem or concern to a more manageable number of product lots (which then would be tracked and investigated).

As non-limiting examples, the disclosed embodiments are directed to three scenarios: (1) the situation where lots or cases are unable to be consistently arranged on or pulled from a pallet, and therefore random sampling is assumed, (2) the situation where lots or cases are known to be strictly pulled in a first-in-first-out (FIFO) manner from one or more pallets, and (3) a hybrid situation where either random or FIFO selection occurs depending on the sequence of events occurring in the warehouse slot.

In some embodiments, the disclosed and/described approach may be implemented by the following set of steps, stages, functions, processes, or operations (which may be applied iteratively to generate updated values as cases/lots are removed or added to a pallet/storage/retail location, for the example of a random sampling or selection process:
Determine current status of product in warehouse slots awaiting distribution through fulfillment of one or more orders. This may include;
   A number (n) of cases of a food product or ingredient currently being stored in a location (such as a “pick slot”) from which an order will be fulfilled;
      Note that this may be obtained from inventory transaction records of a WMS for each slot location;
   For each of one or more lot identifiers (referred to as batch lot codes) in a slot, determine the number of cases that have the specified batch lot code (i.e., Cᵢ as a function of a specific batch lot, i) and are stored in (or on) the pallet or location serving as the source for the cases or lots expected to be used to fulfill an order;
Identify the number of cases of the food product or ingredient currently being stored in a location to select as part of fulfilling an order—this value is (p);
   In one situation, this value may be determined by consideration of the number of cases needed for the order and the number of cases or lots available at the location (i.e., the pallet or storage location);
   This value may be impacted or determined based on consideration of level of service obligations provided to the recipient of the order (such as a priority for receipt of certain items);
Based on the determined values (i.e., at time t), calculate the following for each outgoing sales order line item:

$$Batch lot proportion = {T}_{i} = ( {C}_{i} / n ) ;$$

This is the proportion of the total cases in the sales order with a specified batch lot (i);

$$Batch lot cases = {S}_{i} = {T}_{i} * p$$

This is the number of equivalent cases in the sales order with the specified batch lot (i);

$$Batch lot probability = {P}_{i} = 1 - {( ( n - {C}_{i} ) / n )}^{p}$$

This is the probability that a specified lot (i) was selected for fulfilling the order;
If desired, provide the calculated values of Tᵢ, Sᵢ, and Pᵢ as inputs to a tracking report of the distribution of batch lots and lot sources and to assist in determining the probability of a specific batch lot being used to fulfill the order;
   This information may be used to assist in tracing the source of a food product or ingredient of concern during an outbreak of a foodborne illness (i.e., batch lot and lot source corresponding to the food ingredient or product);
Iteratively update the values of n and Cᵢ over time, based on removal from and/or addition of cases and lots to a slot, where the updated values of (n) and (Cᵢ) over time are noted as (t+1):

$${n}_{t} \to {n}_{t + 1} ; {C}_{i , t} \to {C}_{i , t + 1}$$

Represent a change to the values of nₜ and C_{i,t} after removal of one or more cases/lots from the slot by;

$$Cases available in slot = {n}_{t + 1} = {n}_{t} - p$$

$$Cases per batch lot = {C}_{i , t + 1} = {C}_{i , t} - {S}_{i} ;$$

Represent a change to the values of nₜ and C_{i,t} after addition of one or more cases/lots to the slot by;

$$Cases available in slot = {n}_{t + 1} = {n}_{t} + q$$

$$Cases per batch lot = {C}_{i , t + 1} = {C}_{i , t} + {Q}_{i} ,$$

where q is the transaction case quantity (i.e., the number of cases of a food product or ingredient added to the slot location) and Q is the transaction case quantity per batch lot (i.e., the number of cases having a specific batch lot code, i); and
Repeat the previous steps or stages as purchase orders are received and sales orders are fulfilled to generate a set of updated values for Tᵢ, Sᵢ, and Pᵢ to provide to an entity and/or store in the final mile of the supply chain via a data management system.

Other objects and advantages of the systems, apparatuses, and methods disclosed will be apparent to one of ordinary skill in the art upon review of the detailed description and the included figures. Throughout the drawings, identical reference characters and descriptions indicate similar, but not necessarily identical, elements. While the embodiments disclosed or described herein are susceptible to various modifications and alternative forms, specific embodiments are shown by way of example in the drawings and are described in detail herein. However, embodiments of the disclosure are not limited to the exemplary or specific forms described. Rather, the disclosure covers all modifications, equivalents, and alternatives falling within the scope of the appended claims.

### BRIEF DESCRIPTION OF THE DRAWINGS

Embodiments of the disclosure are described with reference to the drawings, in which:

FIG. 1(a) is an illustration of a random sampling or selection model that may be implemented as part of the disclosed approach, in accordance with some embodiments;

FIG. 1(b) is an illustration of a first-in-first-out (FIFO) selection model that may be implemented as part of the disclosed approach, in accordance with some embodiments;

FIG. 1(c) is a flow chart or flow diagram illustrating a process, operation, method, or set of functions for implementing a method to trace a case or lot code of a food product for the example of a random sampling or selection process, in accordance with some embodiments;

FIG. 1(d) is a flow chart or flow diagram illustrating a process, operation, method, or set of functions for implementing a method to trace a case or lot code of a food product for the example of a first-in-first-out (FIFO) selection process, in accordance with some embodiments;

FIG. 1(e) (and sub-FIGS. 1(e)-A to 1(e)-D) is an illustration of a hybrid selection model, indicating the sequence of events that trigger either random or FIFO selection;

FIG. 1(f) is an illustration of an example flow of data into and within a warehouse management system (WMS), and that may be used as a source of data and information in an embodiment;

FIG. 2 is a diagram illustrating elements or components that may be present in a computer device or system configured to implement a method, process, function, or operation in accordance with some embodiments; and

FIGS. 3, 4, and 5 are diagrams illustrating an architecture for a multi-tenant or Saas platform that may be used in implementing an embodiment of the systems, apparatuses, and methods disclosed and/or described herein.

### DETAILED DESCRIPTION

One or more embodiments of the disclosed subject matter are described herein with specificity to meet statutory requirements, but this description does not limit the scope of the claims. The claimed subject matter may be embodied in other ways, may include different elements or steps, and may be used in conjunction with other existing or later developed technologies. The description should not be interpreted as implying any required order or arrangement among or between various steps or elements except when the order of individual steps or arrangement of elements is explicitly noted as being required.

Embodiments of the disclosed subject matter are described more fully herein with reference to the accompanying drawings, which show by way of illustration, example embodiments by which the disclosed systems, apparatuses, and methods may be practiced. However, the disclosure may be embodied in different forms and should not be construed as limited to the embodiments set forth herein; rather, these embodiments are provided so that the disclosure will satisfy the statutory requirements and convey the scope of the disclosure to those skilled in the art.

Among other forms, the subject matter of the disclosure may be embodied in whole or in part as a system, as one or more methods, or as one or more devices. Embodiments may take the form of a hardware implemented embodiment, a software implemented embodiment, or an embodiment combining software and hardware aspects. For example, in some embodiments, one or more of the operations, functions, processes, or methods disclosed and/or described herein may be implemented by a suitable processing element or elements (such as a processor, microprocessor, CPU, GPU, TPU, QPU, state machine, or controller, as non-limiting examples) that are part of a client device, server, network element, remote platform (such as a SaaS platform), an “in the cloud” service, or other form of computing or data processing system, device, or platform. In some embodiments, data regarding batch lots, lot source, and the calculated probabilities disclosed herein may be stored on and accessed using a blockchain or similar mechanism.

The processing element or elements may be programmed with a set of executable instructions (e.g., software instructions), where the instructions may be stored on (or in) one or more suitable non-transitory computer-readable data storage elements. In some embodiments, the set of instructions may be conveyed to a user over a network (e.g., the Internet) through a transfer of instructions or an application that executes a set of instructions.

In some embodiments, the systems and methods disclosed and/or described herein may provide services or functionality through a SaaS or multi-tenant platform. The platform provides access to multiple entities, each with a separate account and associated data storage. Each account may correspond to an individual, a warehouse, a food product or ingredient producer, a regulatory or administrative agency, an industry, or an organization, for example. Each account may access one or more services, a set of which are instantiated in their account, and which implement one or more of the methods or functions disclosed and/or described herein.

In some embodiments, one or more of the operations, functions, processes, or methods described herein may be implemented by a specialized form of hardware, such as a programmable gate array, application specific integrated circuit (ASIC), or the like. Note that an embodiment of the disclosed and/or described methods may be implemented in the form of an application, a sub-routine that is part of a larger application, a “plug-in”, an extension to the functionality of a data processing system or platform, or other suitable form. The following detailed description is, therefore, not to be taken in a limiting sense.

In one embodiment, the inventors treated tracing a lot of a food product or ingredient used to fulfill an order or purchase as a variation of the “urn” model used to simulate selecting items from a population and approximating the results of the selection process. In this probability problem, there is an urn with different colored marbles. For a selection from the urn one can apply probability relationships to answer questions about the sample selected from the urn. These questions might include “What are the expected colors in the sample?”, “What is the chance that the sample contains a specific color?”, “What if we change the number of marbles in the urn?”, or “What if we repeat the process many times?”, as examples of information that may be determined or at least approximated.

Connecting this to the traceability problem, assume the urn represents a unique warehouse slot or retail shelf (such as a specific storage location containing a pallet, pallets, cases or items) where a particular product is assigned. A pallet containing cases from one or more batch lots is placed into a slot to fulfill orders. The marbles represent individual cases of that product, and the colors of the marbles represent different batch lots (assumed to be from a lot source).

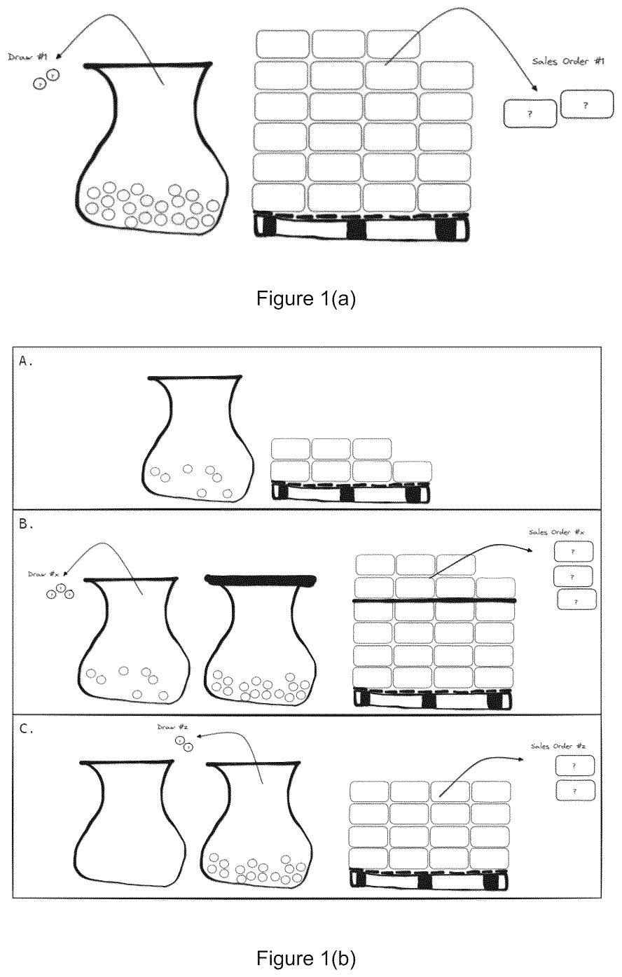

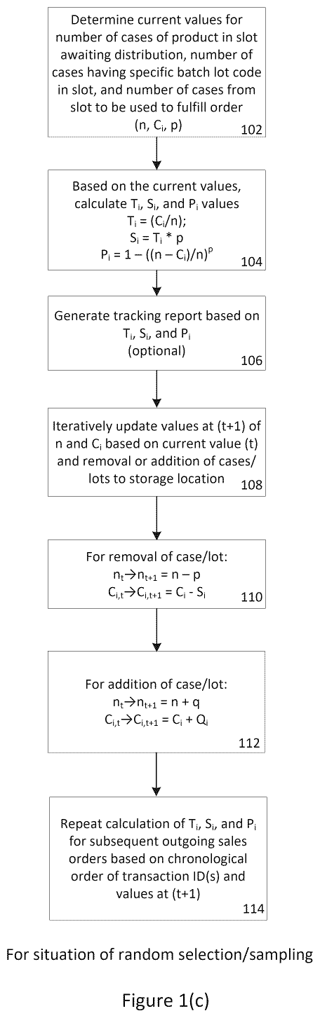

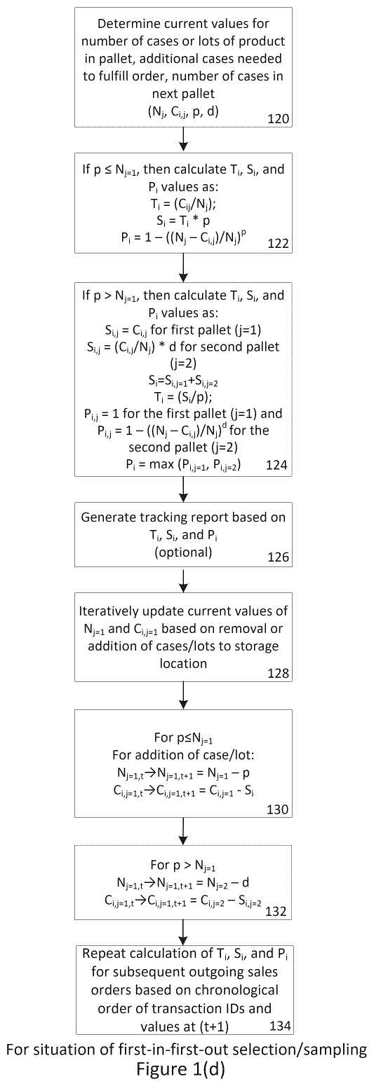

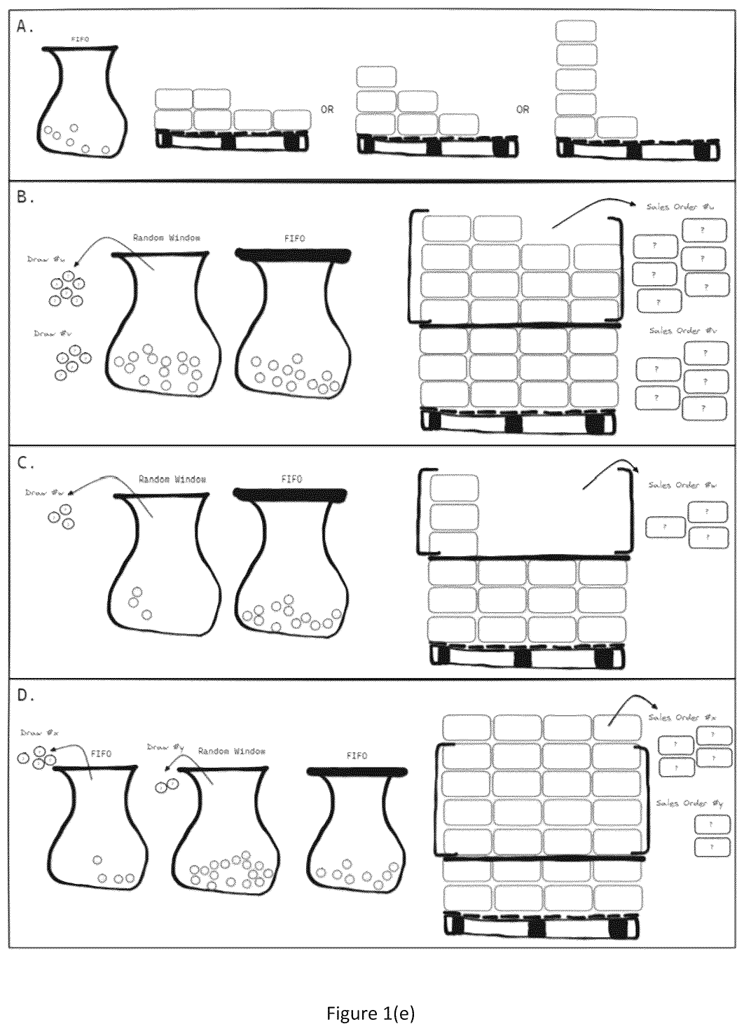

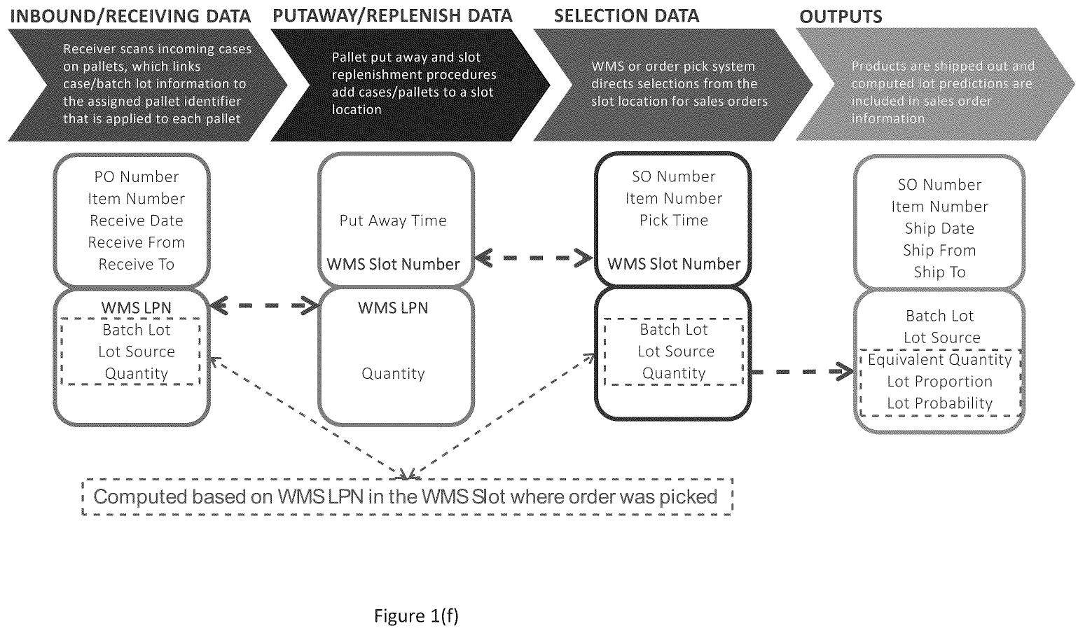

The reader is referred to FIG. 1(a), which is an illustration of a random sampling or selection model that may be implemented as part of the disclosed approach, in accordance with some embodiments. As shown in the figure, in this approximation, a selection of marbles from the urn is treated as a random sampling or selection of one or more batch lot codes from a pallet in a slot or other storage location.

Note that unlike a marble, the location of a case on a pallet is static and is most likely arranged next to other cases from the same batch lot; however, this information is not known (or at least not precisely enough to rely upon) and can therefore be assumed to be random for modeling purposes. The warehouse management system (WMS) directs a sales order to be fulfilled from this pallet/slot via a selection transaction and under the new FDA/FSMA regulation, the batch lot and lot source (i.e. origin information) are required to be included in the shipment information for the items selected.

New pallets of cases may be added to the slot as needed (referred to as “put away” or “replenishment”) to ensure that order fulfillment occurs efficiently over time. Typically, new cases/lots are added to the bottom of a stack and previously leftover cases (those not used to fulfill previous orders) are placed on top of the newly added ones. However, it is possible that new or returned items get placed on top of the stack.

With reference to the urn and marble analogy, new marbles with different colors are continuously added to the urn, and potentially before the urn has been completely depleted of its contents. The replenishment and selection processes, combined with poor visibility of batch lots, makes tracing outgoing batch lots on cases a particular challenge for both warehouses that distribute food products or ingredients and retailers that sell food products or ingredients. This complicates determining what batch lots are present in a slot when a sales order is filled, what batch lots were selected to fulfill a sales order, and what is the likelihood that a sales order contains a specific batch lot present in the slot, among other difficulties.

In one embodiment (as a worst case, conservative approach), it is assumed that there are multiple batch lots on a pallet, and there is no prior information regarding how batch lots are distributed among the cases. Therefore, the process of selecting cases from the slot to fulfill a sales order is treated as equivalent to a random sampling operation (without replacement), where every batch lot has a chance of being selected. For each sampling operation, the proportion of cases from each batch lot (“batch lot proportion”) and the chance or probability of selecting at least one case from each batch lot (“batch lot probability”) can be calculated. Using the batch lot proportion and the number of cases in an order, an estimated number of cases from each batch lot (“equivalent batch lot cases”) can then be calculated.

In one embodiment, the disclosed and/or described approach relies on an initial value of the inventory available in a warehouse slot and then considers one or more unique transactions occurring to that inventory over time. To simulate a random sampling and calculate the results for a selection/transaction, the following inputs are utilized:
Total number of cases in a slot, expressed as (n);
Number of cases in the slot per batch lot, expressed as (Cᵢ), where “i” represents a specific batch lot identifier;
Number of cases to select from the slot for fulfillment of a sales order/transaction, expressed as (p).

Initially, the total number of cases in a slot (n) is derived from WMS inventory transaction records and the number of cases per batch lot (Cᵢ) (and the associated lot source) is derived from WMS receiving and storage transaction records for the cases currently in a slot.

To simulate fulfillment of a sales order for an item at time (t) (as represented by a case with a specific batch lot code), the disclosed and/or described approach simulates a selection transaction from a slot and generates the required output (i.e., the corresponding traceability data for each case used to fulfill an order) per batch lot based on the following relationships:

$$Batch lot proportion = {T}_{i} = ( {C}_{i} / n ) \qquad ( Eq . 1 )$$

$$Batch lot cases = {S}_{i} = {T}_{i} * p \qquad ( Eq . 2 )$$

$$Batch lot probability = {P}_{i} = 1 - {( ( n - {C}_{i} ) / n )}^{p} \qquad ( Eq . 3 )$$

where Tᵢ is the proportion of the total cases in the order with a specified batch lot (i), S, is the number of equivalent cases in the order with the specified batch lot (i), and Pᵢ is the probability that a specific batch lot (i) present in the slot is selected for fulfilling the order.

After each selection transaction or order fulfillment, the approach uses the number of cases in the order (p) to update the slot inventory/contents for the next transaction (nₜ₊₁) and the batch lot cases estimated (Sᵢ) to update the cases per batch lot (i) left in the slot for the next transaction (C_{i,t+1}):

$$Cases available in slot = {n}_{t + 1} = {n}_{t} - p \qquad ( Eq . 4 )$$

$$Cases per batch lot = {C}_{i , t + 1} = {C}_{i , t} - {S}_{i} \qquad ( Eq . 5 )$$

For an addition or replenishment transaction that adds to the number of cases/lots in a slot or storage location, the approach uses the transaction case quantity (q) and the transaction case quantity per batch lot (Qᵢ) to update the slot inventory for the next transaction nₜ₊₁ and the cases per batch lot (i) for the next transaction C_{i,t+1}:

$$Cases available in slot = {n}_{t + 1} = {n}_{t} + q \qquad ( Eq . 6 )$$

$$Cases per batch lot = {C}_{i , t + 1} = {C}_{i , t} + {Q}_{i} \qquad ( Eq . 7 )$$

FIG. 1(c) is a flow chart or flow diagram illustrating a process, operation, method, or set of functions for implementing a method to trace a lot code of a food product for the example of random sampling, in accordance with some embodiments. As shown in the figure, an iteration of the disclosed and/or described process may include:
Accessing, obtaining, or otherwise determining the current values for the number of cases or lots of a food product or ingredient in a slot or storage location (n), the number of cases having a specific batch lot code in the slot (Cᵢ), and the number of cases from the slot to be used to fulfill a sales order (p) (as suggested by step or stage 102);
Based on the current values (i.e., at time t), calculate the quantities Tᵢ, Sᵢ, and Pᵢ (as suggested by step or stage 104), where:

$$\begin{matrix}{T}_{i} = ( {C}_{i} / n ) ; \\ {S}_{i} = {T}_{i} * p ; \\ {P}_{i} = 1 - {( ( n - {C}_{i} ) / n )}^{p} ;\end{matrix}$$

Generate a tracking report based on Tᵢ, Sᵢ, and Pᵢ (optional, and as suggested by step or stage 106);
Iteratively update the values of n and Cᵢ over time (t+1) based on current values (t) and the removal or addition of cases/lots to a pallet or storage location (as suggested by step or stage 108);
   For the removal of a case or lot from the slot (as suggested by step or stage 110)

$$\begin{matrix}{n}_{t} \to {n}_{t + 1} = {n}_{t} - p \\ {C}_{i , t} \to {C}_{i , t + 1} = {C}_{i , t} - {S}_{i} ;\end{matrix}$$

For the addition of a case or lot to the slot (as suggested by step or stage 112)

$$\begin{matrix}{n}_{t} \to {n}_{t + 1} = {n}_{t} + q \\ {C}_{i , t} \to {C}_{i , t + 1} = {C}_{i , t} + {Q}_{i} ;\end{matrix}$$

Repeat the calculation of Tᵢ, Sᵢ, and Pᵢ for subsequent outgoing sales orders based on chronological order of transactions identifiers and the values at (t+1) (as suggested by step or stage 114)
This may be performed by writing the values of (n, Cᵢ) at (t+1) to the location where the values at (t) were accessed from in step or stage 102.

An alternative approach to the random selection simulation is to assume that the warehouse or retailer operates under a strict policy to fulfill sales orders on a first-in, first-out (FIFO) basis based on the order in which cases/lots are placed into a slot or shelf (assuming that the order in which they are placed in the slot aligns with the chronological order of manufacturing or expiration dates). In this alternative, the urn-marble analogy is modified so that instead of adding more marbles to the same urn, a replenishment step is treated as adding another closed urn whose contents are not available for selection until the previous urn is emptied.

This approach is illustrated in FIG. 1(b), which is an illustration of a first-in-first-out (FIFO) selection model that may be implemented as part of the disclosed and/or described approach, in accordance with some embodiments. This scenario removes the random selection of a case and instead introduces a prioritization step based on the order in which batch lots on pallets are placed into a slot.

As shown in FIG. 1(b), the urn and marbles random sampling model may be modified to account for a FIFO selection procedure from a warehouse slot. At the time of replenishment (FIG. 1(b)-B), new items are added but are not available for selection until the previous items (FIG. 1(b)-A) have been completely removed. At the point when the previous items have been depleted (FIG. 1(b)-C), the approach continues selecting from the next urn/pallet.

To simulate a FIFO workflow, the approach prioritizes the oldest pallet (and corresponding batch lots) in the slot as the pallet that will be selected from and depleted first, denoted as j=1 (FIG. 1(b)-A). Upon an addition or replenishment transaction to the warehouse slot (FIG. 1(b)-B), the approach assigns the pallet as the next priority, j=2, and sets the cases on the pallet (Nⱼ₌₂) as the transaction case quantity (q) and the count of cases per batch lot in the prioritized pallet (C_{i,j=2}) as the transaction case quantity per batch lot (Q) (as in Eq. 6 & 7). When the first prioritized pallet is depleted (FIG. 1(b)-C), the prioritization moves the selection process to the next pallet and continues. To calculate the results for a selection, the following inputs are utilized:
The total number of cases in pallet priority (j), expressed as Nⱼ;
Number of cases per batch lot (i) in the pallet priority (j), expressed as (C_{i,j});
Number of cases to select from the slot for fulfillment of a sales order/transaction, expressed as (p);
The difference between the number of cases to select and the cases in the prioritized pallet, expressed as (d), where d=p−Nⱼ₌₁;

Given the above, the approach to determining the batch lot proportion, batch lot cases, and batch lot probability differs depending on whether the number of cases to select for a sales order (p) is available within the first prioritized pallet cases, Nj. After each selection, the approach uses the number of cases in the order (p) and the estimated batch lot cases (Sᵢ) to update the number of cases in the prioritized pallet (N_{j=1,t+1}) and the cases per batch lot in the prioritized pallet (C_{i,j,t+1}) for the next transaction, respectively.

If p≤Nⱼ₌₁ then:

$$Batch lot proportion = {T}_{i} = ( {C}_{i , j} / {N}_{j} ) \qquad ( Eq . 8 )$$

$$Batch lot cases = {S}_{i} = {T}_{i} * p \qquad ( Eq . 2 )$$

$${Batch lot probability = {P}_{i} = 1 - ( ( {N}_{j} ) - {C}_{i , j} ) / {N}_{j} )}^{p} \qquad ( Eq . 9 )$$

$$Cases in prioritized pallet = {N}_{j = 1 , t + 1} = {N}_{j = 1 , t} - p \qquad ( Eq . 10 )$$

$$Cases per batch lot in prioritized pallet = {C}_{i , j = 1 , t + 1} = {C}_{i , j = 1 , t} - {S}_{i} \qquad ( Eq . 11 )$$

If p>Nⱼ₌₁ then:

$$Batch lot cases = {S}_{i} = {\sum }_{j = 1}^{2} {S}_{i , j} = \{ \begin{matrix}{S}_{i , j} = {C}_{i , j} & for j = 1 \\ {S}_{i , j} = ( \frac{{C}_{i , j}}{{N}_{j}} ) * d & for j = 2\end{matrix} \qquad ( Eq . 12 )$$

$$Batch lot proportion = {T}_{i} ( \frac{{S}_{i}}{p} ) \qquad ( Eq . 13 )$$

$$Batch lot probabilty = {P}_{i} = max ( {P}_{i , j} ) = \{ \begin{matrix}{P}_{i , j} = 1 & for j = 1 \\ {P}_{i , j} = 1 - {( ( {N}_{j} - {C}_{i , j} ) / {N}_{j} )}^{d} & for j = 2\end{matrix} \qquad ( Eq . 14 )$$

$$Cases in prioritized pallet = {N}_{j = 1 , t + 1} = {N}_{j = 2 , t} - d \qquad ( Eq . 15 )$$

$$Cases per batch lot in prioritized pallet = {C}_{i , j = 1 , t + 1} = {C}_{i , j = 2 , t} - {S}_{i , j = 2} \qquad ( Eq . 16 )$$

FIG. 1(d) is a flow chart or flow diagram illustrating a process, operation, method, or set of functions for implementing a method to trace a case or lot code of a food product for the example of a first-in-first-out (FIFO) selection process, in accordance with some embodiments. As shown in the figure, an iteration of the disclosed and/or described process may include:
Accessing, obtaining, or otherwise determining the current values for the following (as suggested by step or stage 120):
   The total number of cases in pallet priority (j), expressed as Nⱼ;
   Number of cases per batch lot (i) in pallet priority (j), expressed as (C_{i,j});
   Number of cases to select from the slot for fulfillment of a sales order/transaction, expressed as (p);
   The difference between the number of cases to select and the cases in the prioritized pallet, expressed as (d), where d=p−Nⱼ₌₁;
Based on the current values (i.e., at time t), and whether the value of (p) is less than or equal to the cases on the first pallet (Nⱼ₌₁), calculate the quantities Tᵢ, Sᵢ, and Pᵢ, where:
   If p≤Nⱼ₌₁, then calculate Tᵢ, Sᵢ, and Pᵢ values as (as suggested by step or stage 122):

$$\begin{matrix}{T}_{i} = ( {C}_{i , j = 1} / {N}_{j = 1} ) ; \\ {S}_{i} = {T}_{i} * p \\ {P}_{i} = 1 - {( ( {N}_{j = 1} - {C}_{i , j = 1} ) / {N}_{j = 1} )}^{p}\end{matrix}$$

If p>Nⱼ₌₁, then calculate Tᵢ, Sᵢ, and Pᵢ values as (as suggested by step or stage 124):

$$\begin{matrix}{S}_{i , j} = {C}_{i , j} for first pallet j = 1 \\ {S}_{i , j} = ( {C}_{i , j} / {N}_{j} ) * d for second pallet j = 2 \\ {S}_{i} = the sum of {S}_{i , j} for j = 1 and j = 2 = {S}_{i , j = 1} + {S}_{i , j = 2} \\ {T}_{i} = ( {S}_{i} / p ) ; \\ {P}_{i , j} = 1 for the first pallet j = 1 and \\ {P}_{i , j} = 1 - {( ( {N}_{j} - {C}_{i , j} ) / {N}_{j} )}^{d} for the second pallet j = 2 \\ {P}_{i} = the maximum of {P}_{i , j} for j = 1 and j = 2 = max ( {P}_{i , j = 1} , {P}_{i , j = 2} )\end{matrix}$$

Generate a tracking report based on Tᵢ, Sᵢ, and Pᵢ (optional, and as suggested by step or stage 126);
Iteratively update current values of Nⱼ₌₁ and C_{i,j=1} based on removal of cases/lots to pallet or storage location (as suggested by step or stage 128);
   For p≤Nⱼ₌₁ (as suggested by step or stage 130):

$$\begin{matrix}{N}_{j = 1 , t} \to {N}_{j = 1 , t + 1} = {N}_{j = 1 , t} - p \\ {C}_{i , j = 1 , t} \to {C}_{i , j = 1 , t + 1} = {C}_{i , j = 1 , t} - Si ;\end{matrix}$$

For p>Nⱼ₌₁ (as suggested by step or stage 132):

$$\begin{matrix}{N}_{j = 1 , t} \to {N}_{j = 1 , t + 1} = {N}_{j = 2 , t} - d \\ {C}_{i , j = 1 , t} \to {C}_{i , j = 1 , t + 1} = {C}_{i , j = 2 , t} - {S}_{i , j = 2} ;\end{matrix}$$

Repeat the calculation of Tᵢ, Sᵢ, and Pᵢ for subsequent outgoing sales orders based on chronological order of transaction IDs and values at (t+1) (as suggested by step or stage 134);
   This may be performed by writing the values of (Nⱼ₌₁, C_{i,j=1}, Nⱼ₌₂, C_{i,j=2}) at (t+1) to the location where the values at (t) were accessed from in step or stage 120.

Recognizing that it is unlikely that a warehouse completely lacks a selection policy (i.e., random selection) or operates under a strict FIFO policy for all products, a more flexible approach is to allow for either random or FIFO selection depending on the nature and sequence of events occurring in the warehouse pick slot. This possibility allows for occasional mixing of cases across pallets (and/or batch lots) especially for products with inconsistent replenishments and a high-volume of selections.

FIG. 1(e) (and sub-FIGS. 1(e)-A to 1(e)-D) is a diagram illustrating updating the urn and marbles random sampling model to account for alternating first in, first out (FIFO) and random selection procedures from a warehouse slot. This is relevant because selection can occur from front-to-back or in a stair-step manner within a slot, as opposed to top-to-bottom (as illustrated in FIG. 1(e)-A), meaning the urns in FIFO are not completely closed off.

Depending on the conditions at the time of replenishment 1(e)-B, there is a “window” of random selection across both urns/pallets before transitioning completely from one pallet to the next. When the random window is set, all selections made for sales orders follow the random selection approach (FIG. 1(e)-B,C). When the sum of cases selected for sales orders exceeds the cases in the random window, the selection method reverts to FIFO until the next random window is triggered by a transition to another urn/pallet (FIG. 1(e)-D).

In this scenario (termed “hybrid” herein), it is assumed that FIFO is the default replenishment and selection method. As such, upon an addition or replenishment transaction to the warehouse slot, the disclosed and/or described algorithm/approach assigns the next priority and sets the cases on the pallet (Nⱼ₌₂) as the transaction case quantity (q) and count of cases per batch lot, i, in the prioritized pallet (C_{i,j}) as the transaction case quantity per batch lot (Qᵢ). When the first prioritized pallet is depleted, prioritization moves to the next pallet and selections continue (as illustrated in FIG. 1(b) and Eq. 6 & 7 for FIFO).

However, in the hybrid approach, there is a “window” of random selection when transitioning from one pallet to the next (as illustrated in FIG. 1(e)-B). The maximum size of the random window is determined based on the fixed pallet size for a product/slot (in industry terms, ‘ti’ and ‘hi’) and could be up to the entire number of cases on a pallet. The composition of cases in the random window at a given time is dependent on the number of cases remaining in the prioritized pallet at the time of replenishment (Nⱼ₌₁), the number of cases to replenish or the transaction quantity (q), and the maximum size of the random window (E).

When the random window is set (rv_flag=1), all selections made for sales orders follow the random selection approach (as illustrated in FIG. 1(e)-B,C). When the sum of cases selected for subsequent sales orders exceeds the cases in the random window, the selection method reverts to FIFO (rv_flag=0) until the next random window is triggered by a transition to another pallet (as illustrated in FIG. 1(e)-D).

By including product-specific parameters, the hybrid scenario likely provides lot code predictions that fall between the random and FIFO scenarios in terms of key traceability metrics: number of sales orders per lot code and number lot codes per sales order.

As mentioned, for the hybrid approach, the default prioritization of items in a slot follows as in FIFO, where priority is given to the oldest pallet (and batch lots) in the slot as the pallet that will be selected from and depleted first, j=1. However, when the first prioritized pallet nears depletion (as illustrated in FIG. 1(e)-A), selections may be taken from both the first and next prioritized pallets in the random window (FIG. 1(e)-B) before moving the priority to the next pallet (and batch lots) (FIG. 1(e)-D).

Upon an addition or replenishment transaction to the warehouse slot (FIG. 1(e)-D), the algorithm assigns the next priority, j=2, and sets the cases on the pallet (Nⱼ₌₂) as the transaction case quantity (q) and count of cases per batch lot, i, in the prioritized pallet (C_{i,j=2}) as the transaction case quantity per batch lot (Q) coming from WMS data (as in Eq. 6 & 7).

Random selection may apply at the end of the prioritized pallet according to the following parameters:
The maximum size (in cases) of the random window for the item/slot (E)
Total number of cases in pallet priorities, j (Nⱼ)
Count of cases per batch lot, i, in the pallet priorities, j (C_{i,j})
Count of cases per batch lot, i, in pallet, j, in the random window (R_{i,j})
The current number of cases in the random selection window

$$( W = {\sum }_{i = 0 , j = 0}^{∞} {R}_{i , j} )$$

Count of cases per batch lot, i, in the random window

$$( {R}_{i} = {\sum }_{j = 0}^{∞} {R}_{i , j} )$$

A Boolean variable indicating random selection (rv_flag=0 for FIFO, rv_flag=1 for random)
Number of cases to select from the slot for a sales order (p)
The difference between the cases to select and the cases in prioritized pallet (d=p−Nⱼ₌₁)
The difference between the cases to select and the cases in random window (f=p−W)

The calculations for model outputs and updating the slot inventory is therefore dependent on whether FIFO or random selection is triggered (rv_flag) and on whether the number of cases to select for a sales order (p) is available within the first prioritized pallet cases, Nⱼ₌₁, or the random window (W), respectively.

If rv_flag=0, then FIFO Selection:
If p≤Nimi then:

$$Batch lot proportion = {T}_{i} = ( \frac{{C}_{i , j = 1}}{{N}_{j = 1}} ) \qquad ( Eq . 8 )$$

$$Batch lot cases = {S}_{i} = {T}_{i} * p \qquad ( Eq . 2 )$$

$$Batch lot probability = {P}_{i} = 1 - {( ( {N}_{j = 1} - {C}_{i , j = 1} ) / {N}_{j = 1} )}^{p} \qquad ( Eq . 9 )$$

If p>Nⱼ₌₁ then:

$$Batch lot cases = {S}_{i} = {\sum }_{j = 1}^{2} {S}_{i , j} = \{ \begin{matrix}{S}_{i , j} = {C}_{i , j} & for j = 1 \\ {S}_{i , j} = ( \frac{{C}_{i , j}}{{N}_{j}} ) * d & for j = 2\end{matrix} \qquad ( Eq . 12 )$$

$$Batch lot proportion = {T}_{i} = ( \frac{{S}_{i}}{p} ) \qquad ( Eq . 13 )$$

$$Batch lot probability = {P}_{i} = max ( {P}_{i , j} ) = \{ \begin{matrix}{P}_{i , j} = 1 & for j = 1 \\ {P}_{i , j} = 1 - {( ( {N}_{j} - {C}_{i , j} ) / {N}_{j} )}^{d} & for j = 2\end{matrix} \qquad ( Eq . 14 )$$

After each FIFO selection, the algorithm uses the estimated batch lot cases (S_{i,j}) to update the number of cases in the pallet priorities (N_{j,t+1}) and cases per batch lot in the pallet priorities (C_{i,j,t+1}) for the next transaction. The algorithm then loops back to determine whether random or FIFO selection continues (Eq. 26-32).

$$Cases in pallet priorities = {N}_{j , t + 1} = {N}_{j , t} - {\sum }_{i = 1}^{∞} {S}_{i , j} \qquad ( Eq . 17 )$$

$$Cases per batch lot in pallet priorities = {C}_{i , j , t + 1} = {C}_{i , j , t} - {S}_{i , j} \qquad ( Eq . 18 )$$

If rv_flag=1 and p≥W, then Modified FIFO Selection:

$$Batch lot cases = {S}_{i} = {\sum }_{j = 1}^{3} {S}_{i , j} = \{ \begin{matrix}{C}_{i , j} & for {R}_{i , j} > 0 \\ ( \frac{{C}_{i , j}}{{N}_{j}} ) * f & for {R}_{i , j} = 0\end{matrix} \qquad ( Eq . 19 )$$

$$Batch lot proportion = {T}_{i} = ( \frac{{S}_{i}}{p} ) \qquad ( Eq . 13 )$$

$$Batch lot probability = {P}_{i} = max ( {P}_{i , j} ) = \{ \begin{matrix}{P}_{i , j} = 1 & for j < 3 \\ {P}_{i , j} = 1 - {( ( {N}_{j} - {C}_{i , j} ) / {N}_{j} )}^{d} & for j = 3\end{matrix} \qquad ( Eq . 20 )$$

After each FIFO selection, the algorithm uses the estimated batch lot cases (S_{i,j}) to update the number of cases in the pallet priorities (N_{j,t+1}) and cases per batch lot in the pallet priorities (C_{i,j,t+1}) for the next transaction. The algorithm then loops back to determine whether random or FIFO selection continues (Eq. 26-32).

$$Cases in pallet prorities = {N}_{j , t + 1} = {N}_{j , t} - {\sum }_{i = 1}^{∞} {S}_{i , j} \qquad ( Eq . 17 )$$

$$Cases per batch lot in pallet priorities = {C}_{i , j , t + 1} = {C}_{i , j , t} - {S}_{i , j} \qquad ( Eq . 18 )$$

If rv_flag=1 and p<W, then Random Selection:

$$Batch lot proportion = {T}_{i} = {\sum }_{j = 1}^{∞} {T}_{i , j} = {\sum }_{j = 1}^{∞} ( \frac{{R}_{i , j}}{W} ) \qquad ( Eq . 21 )$$

$$Batch lot cases = {S}_{i} = {\sum }_{j = 1}^{∞} {S}_{i , j} = {\sum }_{j = 1}^{∞} ( {T}_{i , j} * p ) \qquad ( Eq . 22 )$$

$$Batch lot probability = {P}_{i} = 1 - {( ( W - {R}_{i} ) / W )}^{p} \qquad ( Eq . 23 )$$

After each selection from the random window, the algorithm uses the number of cases in the order (p) and the estimated batch lot cases (Sᵢ) to update the number of cases in the random window (W) and then moves directly to the next WMS transaction without having to determine random selection (Eq. 26-32).

$$Cases in pallet prorities = {N}_{j , t + 1} = {N}_{j , t} - {\sum }_{i = 1}^{∞} {S}_{i , j} \qquad ( Eq . 17 )$$

$$Cases per batch lot in pallet priorities = {C}_{i , j , t + 1} = {C}_{i , j , t} - {S}_{i , j} \qquad ( Eq . 18 )$$

$$Cases in random selection window = {W}_{t + 1} = {W}_{t} - p \qquad ( Eq . 24 )$$

$$Cases per batch lot in random window = {R}_{i , t + 1} = {R}_{i , t} - {S}_{i} \qquad ( Eq . 25 )$$

### Determining Random Selection:

The logic for determining when to switch to random selection (rv_flag), and calculate the random window, depends on the number of cases remaining in the prioritized pallet (Nⱼ₌₁), the number of cases to replenish or the transaction quantity (q=Nⱼ₌₂), and the maximum size of the random window (E).

$$If {N}_{j = 1} > 1 / 2 E , then$$

$$rv\_flag = 0 \qquad ( Eq . 26 )$$

$${R}_{i , j} = 0 \qquad ( Eq . 27 )$$

$$If {N}_{j = 1} \leq 1 / 2 E ,$$

$$If {N}_{j = 2} > 1 / 2 E , then$$

$$rv\_flag = 1 \qquad ( Eq . 28 )$$

$${R}_{i , j} = {C}_{i , j} \qquad ( Eq . 29 )$$

$$If {N}_{j = 2} > 1 / 2 E , then$$

$$rv\_flag = 1 \qquad ( Eq . 28 )$$

$${R}_{i , j} = \{ \begin{matrix}{C}_{i , j} & for j = 1 \\ \frac{E}{2} * \frac{{C}_{i , j}}{{N}_{j}} & for j = 2\end{matrix} \qquad ( Eq . 30 )$$

$$W = {\sum }_{i = 0 , j = 0}^{∞} {R}_{i , j} \qquad ( Eq . 31 )$$

$${R}_{i} = {\sum }_{j = 0}^{∞} {R}_{i , j} \qquad ( Eq . 32 )$$

FIG. 1(f) is an illustration of an example flow of data into and within a warehouse management system (WMS), and that may be used as a source of data and information in an embodiment. The inbound order and receiving data provide the batch lot, lot source, and case quantity information and connects it to a pallet (with a unique identifier, e.g., license plate number, LPN) that is then placed in a specific location (also with a unique identifier, e.g., slot number). For a slot number, the WMS inventory transaction data provides chronological transactions that increment or decrement the quantity of items (e.g., replenishments and selections). Thus, the disclosed and/or described approach establishes slots for simulation based on the inbound/receiving data and incremental transactions and then applies the equations for each decremental transaction based on the transaction identifier to provide the batch lot predictions.

A result of applying an embodiment of the disclosed and/or described approach is to provide a calculation of the batch lots likely to be contained in the cases selected for an outgoing sales order to a final customer in the food supply chain. More specifically, the disclosed and/or described calculations provide the expected number of cases per batch lot (and the associated lot source), the proportion of the cases in an order associated with each batch lot, and the probability that a specific batch lot was sent to the customer. This enables full (or at least better) compliance with the new requirements of the FSMA 204 Food Traceability Rule and provides more visibility during an outbreak investigation into where products or ingredients are sourced from and where they are consumed.

In addition, other potential uses of the generated data and information may include:
The data and its implications can be shared with supply chain partners;
The data can be stored in a blockchain to enable secure transfer and access;
The data can be compiled in a sortable spreadsheet for a product during a specified time period (this is expected to be needed for compliance with the 204 regulation).

Based on the generated data and information, one or more of the following may occur:
The scope of a recall or investigation may be widened or preferably narrowed to expedite removal of implicated product and prevent additional consumer illness;
One or more of warehouse and retail receiving, replenishment, and selection activities may be modified to optimize for traceability and product freshness;
The supply chain sourcing may be optimized to reduce mixed lot pallets;
The supply chain sourcing may be optimized for product freshness.

By simulating inventory events and states, an embodiment may be used to refine or create new metrics related to warehouse layout/utilization, inventory status and management, and order processing and management. As a further example, batch lots with relatively high probabilities for products that have both microbiological/diagnostic and epidemiological links to illnesses would be tracked for investigation/removal from stores first before moving to batch lots with lower probabilities. This is an area that is under consideration and development by the FDA and supply chains.

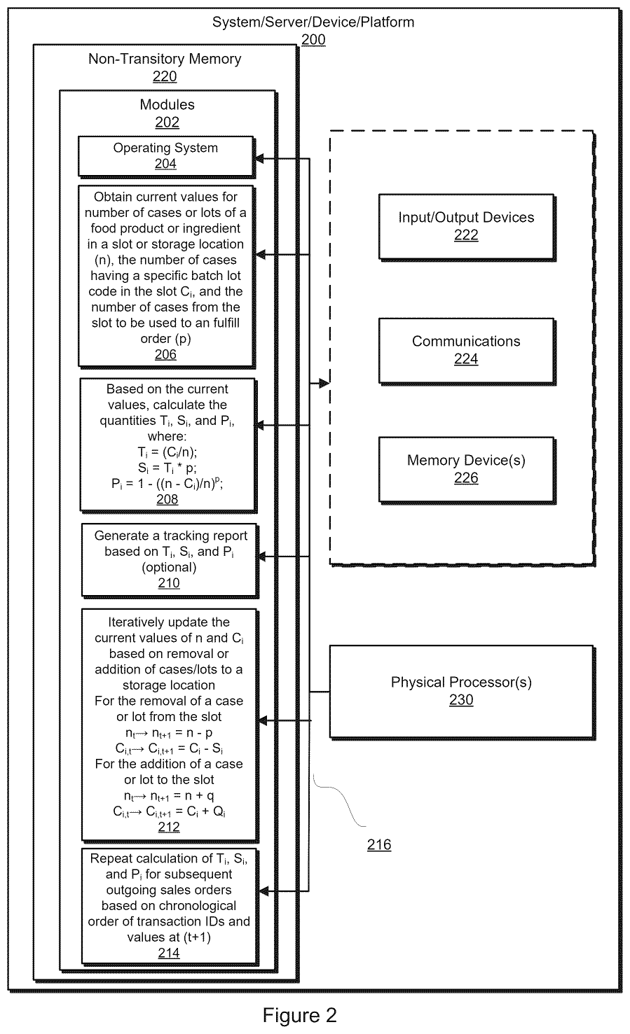

FIG. 2 is a diagram illustrating elements or components that may be present in a computer device or system 200 configured to implement a method, process, function, or operation in accordance with some embodiments. As noted, in some embodiments, the disclosed and/or described system and methods may be implemented in the form of an apparatus that includes a processing element and set of computer-executable instructions. The executable instructions may be part of a software application and arranged into a software architecture.

In general, an embodiment may be implemented using a set of software instructions that are executed by a suitably programmed processing element (such as a GPU, CPU, microprocessor, processor, controller, state machine, or computing device, as non-limiting examples). In a complex application or system such instructions are typically arranged into “modules” with each such module (or sub-module) typically performing a specific task, process, function, or operation. The entire set of modules may be controlled or coordinated in their operation by an operating system (OS) or other form of organizational platform.

Each application module or sub-module may correspond to a particular function, method, process, or operation that is implemented by the module or sub-module. Such function, method, process, or operation may include those used to implement one or more aspects of the disclosed and/or described system and methods.

The application modules and/or sub-modules may include a suitable computer-executable code or set of instructions (e.g., as would be executed by a suitably programmed processor, microprocessor, or CPU), such as computer-executable code corresponding to a programming language. For example, programming language source code may be compiled into computer-executable code. Alternatively, or in addition, the programming language may be an interpreted programming language such as a scripting language.

Modules 202 may contain one or more sets of instructions for performing a method or function described with reference to the Figures, and the disclosure and/or descriptions of the functions and operations provided in the specification. These modules may include those illustrated but may also include a greater number or fewer number than those illustrated.

As mentioned, each module may contain a set of computer-executable instructions. The set of instructions may be executed by a programmed processor contained in a server, client device, network element, system, platform, or other component. The computer-executable instructions that are contained in the modules or in a specific module or sub-module may be executed by the same processor or by different processors. Further, the computer-executable instructions that are contained in a single module may be executed (in whole or in part) by one processor or by more than one processor.

A module or sub-module may contain instructions that are executed by a processor contained in more than one of a server, client device, network element, system, platform, or other component. In some embodiments, a plurality of electronic processors, with each being part of a separate device, server, or system may be responsible for executing all or a portion of the software instructions contained in an illustrated module. Thus, although FIG. 2 illustrates a set of modules which taken together perform multiple functions or operations, these functions or operations may be performed by different devices or system elements, with certain of the modules (or instructions contained in those modules) being associated with those devices or system elements.

As shown in FIG. 2, system 200 may represent a server or other form of computing or data processing system, platform, or device. Modules 202 each contain a set of computer-executable instructions, where when the set of instructions is executed by a suitable electronic processor or processors (such as that indicated in the figure by “Physical Processor(s) 230”), system (or server, platform, or device) 200 operates to perform a specific process, operation, function, or method.

Modules 202 are stored in a (non-transitory) memory 220, which typically includes an Operating System module 204 that contains instructions used (among other functions) to access and control the execution of the instructions contained in other modules. The modules 202 stored in memory 220 are accessed for purposes of transferring data and executing instructions by use of a “bus” or communications line 216, which also serves to permit processor(s) 230 to communicate with the modules for purposes of accessing and executing a set of instructions.

Bus or communications line 216 also permits processor(s) 230 to interact with other elements of system 200, such as input or output devices 222, communications elements 224 for exchanging data and information with devices external to system 200, and additional memory devices 226.

In some embodiments, the modules 202 may comprise computer-executable software instructions that when executed by one or more electronic processors cause the processors or a system or apparatus containing the processors to perform one or more of the steps or stages of (for the situation of a random selection of cases or lots from a storage location, as described with reference to FIG. 1(c)):
Accessing, obtaining, or otherwise determining the current values for the number of cases or lots of a food product or ingredient in a slot or storage location (n), the number of cases having a specific batch lot code in the slot (Cᵢ), and the number of cases from the slot to be used to fulfill a sales order (p) (as suggested by module 206);
Based on the current values (i.e., at time t), calculate the quantities Tᵢ, Sᵢ, and Pᵢ (as suggested by module 208), where:

$${T}_{i} = ( {C}_{i} / n ) ;$$

$${S}_{i} = {T}_{i} * p ;$$

$${P}_{i} = 1 - {( ( n - {C}_{i} ) / n )}^{p} ;$$

Generate a tracking report based on Tᵢ, Sᵢ, and Pᵢ (optional, and as suggested by step or module 210);
Iteratively update the values of n and Cᵢ over time (t+1) based on current values (t) and the removal or addition of cases/lots to a pallet or storage location (as suggested by module 212);
   For the removal of a case or lot from the slot:

$${n}_{t} \to {n}_{t + 1} = {n}_{t} - p$$

$${C}_{i , t} \to {C}_{i , t + 1} = {C}_{i , t} - {S}_{i} ;$$

For the addition of a case or lot to the slot:

$${n}_{t} \to {n}_{t + 1} = {n}_{t} + q$$

$${C}_{i , t} \to {C}_{i , t + 1} = {C}_{i , t} + {Q}_{i} ;$$

Repeat the calculation of Tᵢ, Sᵢ, and Pᵢ for subsequent outgoing sales orders based on chronological order of transaction IDs and values at (t+1) (as suggested by module 214);
   This may be performed by writing the values of (n, Cᵢ) at (t+1) to the location where the values at (t) were accessed from under the control of module 206.

As noted, the description of the instructions in each of the modules is based on the situation of a random selection of cases or lots from a storage location. If instead, a first-in-first-out (FIFO) selection process is used, then the modules would contain instructions for implementing the corresponding operations or functions (i.e., those described with reference to FIG. 1(d)). Similarly, if a hybrid selection process is used, then then the modules would contain instructions for implementing the corresponding operations or functions (i.e., those described with reference to FIG. 1(e) and the associated formulas).

In some embodiments, the functionality and services provided by the system, apparatuses, and methods disclosed and/or described herein may be made available to multiple users by accessing an account maintained by a server or service platform. Such a server or service platform may be termed a form of Software-as-a-Service (Saas). Each account may correspond to an individual, a warehouse, a food product or ingredient producer, a regulatory or administrative agency, an industry, or an organization, for example.

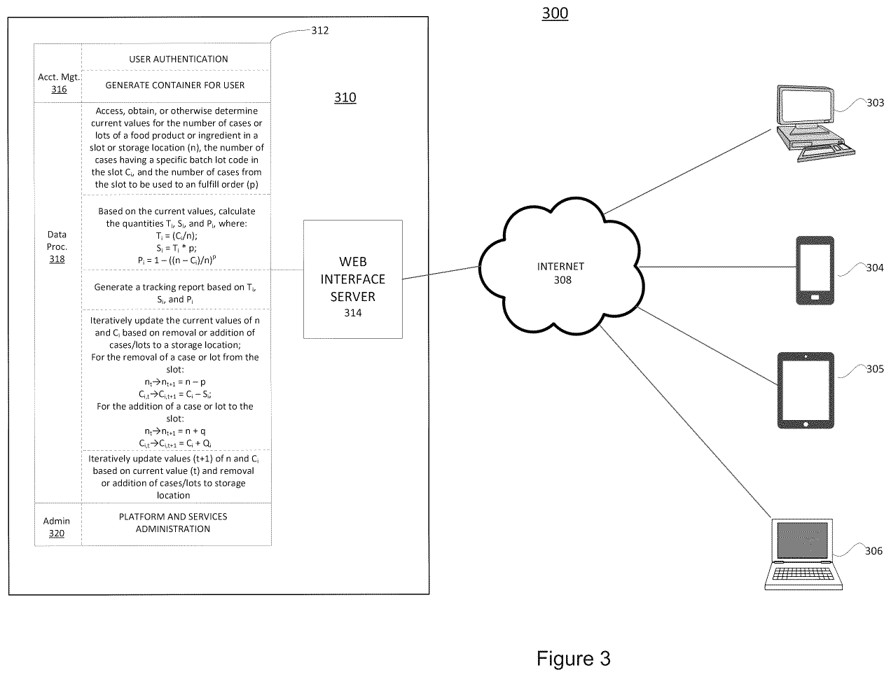

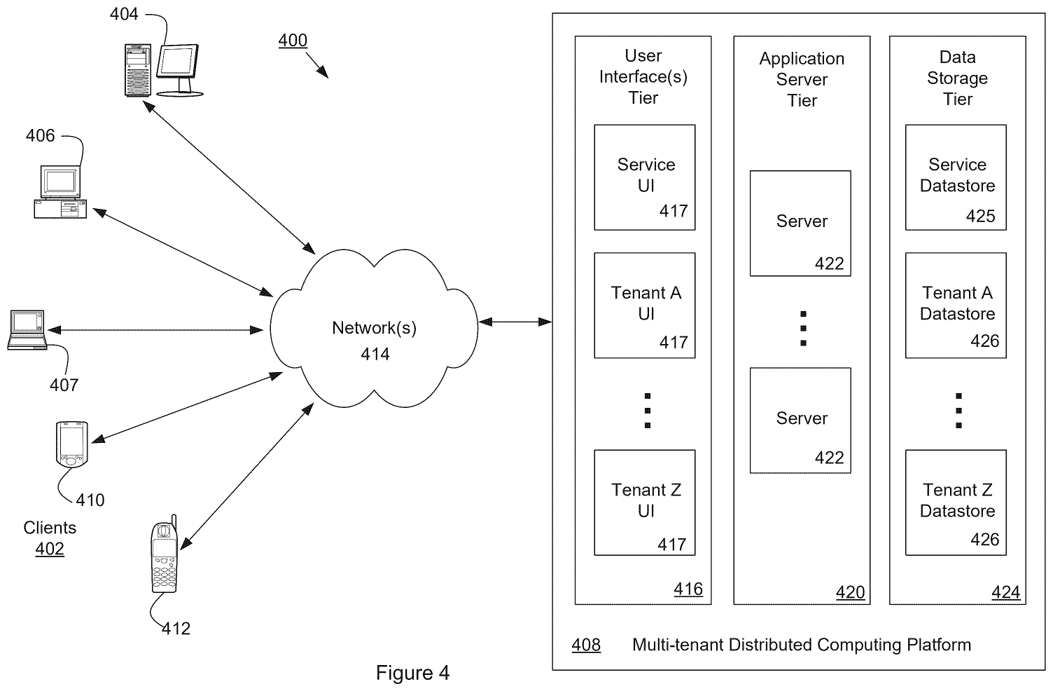

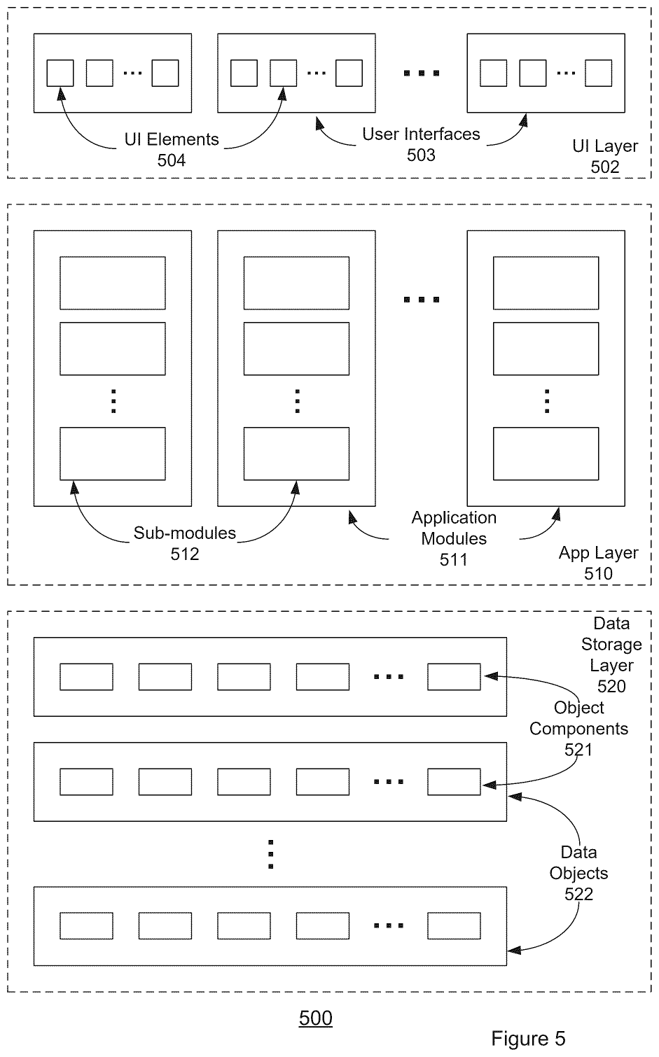

FIGS. 3, 4, and 5 are diagrams illustrating an architecture for a multi-tenant or Saas platform that may be used in implementing an embodiment of the systems, apparatuses, and methods. FIG. 3 is a diagram illustrating a SaaS system with which an embodiment may be implemented. FIG. 4 is a diagram illustrating elements or components of an example operating environment with which an embodiment may be implemented. FIG. 5 is a diagram illustrating additional details of the elements or components of the multi-tenant distributed computing service platform of FIG. 4, with which an embodiment may be implemented.

In some embodiments, the system or services disclosed and/or described herein for assisting in the tracing of a food product or food ingredient to comply with a regulatory requirement may be implemented as microservices, processes, workflows or functions performed in response to the submission of a set of input data. The microservices, processes, workflows or functions may be performed by a server, data processing element, platform, or system.

In some embodiments, the data processing and other services may be provided by a service platform located “in the cloud”. In such embodiments, the platform may be accessible through APIs and SDKs. The functions, processes and capabilities disclosed and/or described herein and with reference to one or more of the Figures may be provided as microservices within the platform. The interfaces to the microservices may be defined by REST and GraphQL endpoints. An administrative console may allow users or an administrator to securely access the underlying request and response data, manage accounts and access, and in some cases, modify the processing workflow or configuration.

Note that although FIGS. 3, 4, and 5 illustrate a multi-tenant or SaaS architecture that may be used for the delivery of business-related or other applications and services to multiple accounts/users, such an architecture may also be used to deliver other types of data processing services and provide access to other applications. For example, such an architecture may be used to provide one or more of the processes, functions, and operations disclosed and/or described herein. Although in some embodiments, a platform or system of the type illustrated in the Figures may be operated by a 3rd party provider to provide a specific set of services or applications, in other embodiments, the platform may be operated by a provider and a different entity may provide the applications or services for users through the platform.

FIG. 3 is a diagram illustrating a system 300 with which an embodiment may be implemented or through which an embodiment of the services described herein may be accessed. In accordance with the advantages of an application service provider (ASP) hosted business service system (such as a multi-tenant data processing platform), users of the services described herein may comprise individuals, businesses, stores, organizations, etc. A user may access the services using a suitable client.

In general, a client device having access to the Internet may be used to provide data to the platform for processing and evaluation. A user interfaces with the service platform across the Internet 308 or another suitable communications network or combination of networks. Examples of suitable client devices include desktop computers 303, smartphones 304, tablet computers 305, or laptop computers 306.

System 310, which may be hosted by a third party, may include a set of data analysis and other services to assist the tracing of a food product or food ingredient to comply with a regulatory requirement 312, and a web interface server 314, coupled as shown in FIG. 3. It is to be appreciated that either or both the data analysis and other services 312 and the web interface server 314 may be implemented on one or more different hardware systems and components, even though represented as singular units in FIG. 3. Services 312 may include one or more functions or operations for the processing of data to assist in the tracing of a food product or food ingredient to comply with a regulatory requirement.

As examples, in some embodiments, the set of functions, operations or services made available through the platform or system 310 may include (for the situation of a random selection of cases or lots from a storage location, as described with reference to FIG. 1(c)):
Account Management services 316, such as:
   a process or service to authenticate a user;
   a process or service to generate a container or instantiation of the data analysis and network evaluation services for that user;
Data Processing services 318, such as a process or service to:
   Access, obtain, or otherwise determine the current values for the number of cases or lots of a food product or ingredient in a slot or storage location (n), the number of cases having a specific batch lot code in the slot (Cᵢ), and the number of cases from the slot to be used to fulfill a sales order (p);
   Based on the current values (i.e., at time t), calculate the quantities Tᵢ, Sᵢ, and Pᵢ, where:

$${T}_{i} = ( {C}_{i} / n ) ;$$

$${S}_{i} = {T}_{i} * p ;$$

$${P}_{i} = 1 - {( ( n - {C}_{i} ) / n )}^{p} ;$$

Generate a tracking report based on Tᵢ, Sᵢ, and Pᵢ (optional);
Iteratively update the values of n and Cᵢ over time (t+1) based on current values (t) and the removal or addition of cases/lots to a pallet or storage location;
   For the removal of a case or lot from the slot:

$${n}_{t} \to {n}_{t + 1} = {n}_{t} - p$$

$${C}_{i , t} \to {C}_{i , t + 1} = {C}_{i , t} - {S}_{i} ;$$

For the addition of a case or lot to the slot:

$${n}_{t} \to {n}_{t + 1} = {n}_{t} + q$$

$${C}_{i , t} \to {C}_{i , t + 1} = {C}_{i , t} + {Q}_{i} ;$$

Repeat the calculation of Tᵢ, Sᵢ, and Pᵢ for subsequent outgoing sales orders based on chronological order of transaction IDs and values at (t+1);
   This may be performed by writing the values of (n, Cᵢ) at (t+1) to the location where the values at (t) were accessed from previously.
Administrative services 320, such as:
   a process or services to provide platform and services administration—for example, to enable the provider of the services and/or the platform to administer and configure the processes and services provided to users.

In addition to the mentioned processes or services, the above may include a data ingestion service that accesses a data source such as server or platform used by a warehouse or distributor or a blockchain. Additional services may also include account management, administrative services, and specific user interface features.

The platform or system shown in FIG. 3 may be hosted on a distributed computing system made up of at least one, but likely multiple, “servers.” A server is a physical computer dedicated to providing data storage and an execution environment for one or more software applications or services intended to serve the needs of the users of other computers that are in data communication with the server, for instance via a public network such as the Internet. The server, and the services it provides, may be referred to as the “host” and the remote computers, and the software applications running on the remote computers being served may be referred to as “clients.” Depending on the computing service(s) that a server offers it could be referred to as a database server, data storage server, file server, mail server, print server, or web server.

As mentioned with regards to FIG. 2, the data processing stages described with regards to FIG. 3 are based on the situation of a random selection of cases or lots from a storage location. If instead, a first-in-first-out (FIFO) selection process is used, then the stages would implement the corresponding operations or functions (i.e., those described with reference to FIG. 1(d)). Similarly, if a hybrid selection process is used, then then the modules would contain instructions for implementing the corresponding operations or functions (i.e., those described with reference to FIG. 1(e) and the associated formulas).

FIG. 4 is a diagram illustrating elements or components of an example operating environment 400 with which an embodiment may be implemented. As shown, a variety of clients 402 incorporating and/or incorporated into a variety of computing devices may communicate with a multi-tenant service platform 408 through one or more networks 414. For example, a client may incorporate and/or be incorporated into a client application (e.g., software) implemented at least in part by one or more of the computing devices.

Examples of suitable computing devices include personal computers, server computers 404, desktop computers 406, laptop computers 407, notebook computers, tablet computers or personal digital assistants (PDAs) 410, smart phones 412, cell phones, and consumer electronic devices incorporating one or more computing device components, such as one or more electronic processors, microprocessors, central processing units (CPU), or controllers. Examples of suitable networks 414 include networks utilizing wired and/or wireless communication technologies and networks operating in accordance with any suitable networking and/or communication protocol (e.g., the Internet).

The distributed computing service/platform (which may also be referred to as a multi-tenant data processing platform) 408 may include multiple processing tiers, including a user interface tier 416, an application server tier 420, and a data storage tier 424. The user interface tier 416 may maintain multiple user interfaces 417, including graphical user interfaces and/or web-based interfaces. The user interfaces may include a default user interface for the service to provide access to applications and data for a user or “tenant” of the service (depicted as “Service UI” in the figure), as well as one or more user interfaces that have been specialized/customized in accordance with user specific requirements (e.g., represented by “Tenant A UI”, . . . , “Tenant Z UI” in the figure, and which may be accessed via one or more APIs).

The default user interface may include user interface components enabling a tenant or platform administrator to administer the tenant's access to and use of the functions and capabilities provided by the service platform. This may include accessing tenant data, launching an instantiation of a specific application, or causing the execution of specific data processing operations.

Each application server or processing tier 422 shown in the figure may be implemented with a set of computers and/or components including computer servers and processors, and may perform various functions, methods, processes, or operations as determined by the execution of a software application or set of instructions. The data storage tier 424 may include one or more data stores, which may include a Service Data store 425 and one or more Tenant Data stores 426. Data stores may be implemented with any suitable data storage technology, including structured query language (SQL) based relational database management systems (RDBMS).

Service Platform 408 may be multi-tenant and may be operated by an entity to provide multiple tenants with a set of business-related or other data processing applications, data storage, and functionality. For example, the applications and functionality may include providing web-based access to the functionality used by a business to provide services to end-users, thereby allowing a user with a browser and an Internet or intranet connection to view, enter, process, or modify certain types of information.

Such functions or applications are typically implemented by one or more modules of software code/instructions that are maintained on and executed by one or more servers 422 that are part of the platform's Application Server Tier 420. As noted with regards to FIG. 3, the platform system shown in FIG. 4 may be hosted on a distributed computing system made up of at least one, but typically multiple, “servers.”

As mentioned, rather than build and maintain such a platform or system themselves, a business may utilize systems provided by a third party. A third party may implement a business system/platform as described above in the context of a multi-tenant platform, where individual instantiations of a business' data processing workflow (such as the data analysis and generation of an optimal bidding strategy services disclosed and/or described herein) are provided to users, with each business representing a tenant of the platform. One advantage to such multi-tenant platforms is the ability for each tenant to customize their instantiation of the data processing workflow to that tenant's specific business needs or operational methods. Each tenant may be a business or entity that uses the multi-tenant platform to provide business services and functionality to multiple users.

FIG. 5 is a diagram illustrating additional details of the elements or components of the multi-tenant distributed computing service platform of FIG. 4, with which an embodiment may be implemented. In general, an embodiment may be implemented using a set of software instructions that are designed to be executed by a suitably programmed processing element (such as a CPU, microprocessor, processor, controller, state machine, or computing device, as non-limiting examples). In a complex system such instructions are typically arranged into “modules” with each such module performing a specific task, process, function, or operation. The entire set of modules may be controlled or coordinated in their operation by an operating system (OS) or other form of organizational platform.

As noted, FIG. 5 is a diagram illustrating additional details of the elements or components 500 of a multi-tenant distributed computing service platform, with which an embodiment may be implemented. The example architecture includes a user interface layer or tier 502 having one or more user interfaces 503. Examples of such user interfaces include graphical user interfaces and application programming interfaces (APIs). Each user interface may include one or more interface elements 504. For example, users may interact with interface elements to access functionality and/or data provided by application and/or data storage layers of the example architecture.

Examples of graphical user interface elements include buttons, menus, checkboxes, drop-down lists, scrollbars, sliders, spinners, text boxes, icons, labels, progress bars, status bars, toolbars, windows, hyperlinks, and dialog boxes. Application programming interfaces may be local or remote and may include interface elements such as parameterized procedure calls, programmatic objects, and messaging protocols.

The application layer 510 may include one or more application modules 511, each having one or more sub-modules 512. Each application module 511 or sub-module 512 may correspond to a function, method, process, or operation that is implemented by the module or sub-module (e.g., a function or process related to providing data processing and services to a user of the platform). Such function, method, process, or operation may include those used to implement one or more aspects of the disclosed and/or described systems and methods, such as for one or more of the processes or functions described with reference to the Figures (for the situation of a random selection of cases or lots from a storage location, as described with reference to FIG. 1(c)):
Access, obtain, or otherwise determine the current values for the number of cases or lots of a food product or ingredient in a slot or storage location (n), the number of cases having a specific batch lot code in the slot (Cᵢ), and the number of cases from the slot to be used to fulfill a sales order (p);
Based on the current values (i.e., at time t), calculate the quantities Tᵢ, Sᵢ, and Pᵢ, where:

$${T}_{i} = ( {C}_{i} / n ) ;$$

$${S}_{i} = {T}_{i} * p ;$$

$${P}_{i} = 1 - {( ( n - {C}_{i} ) / n )}^{p} ;$$

Generate a tracking report based on Tᵢ, Sᵢ, and Pᵢ (optional);
Iteratively update the values of n and C over time (t+1) based on current values (t) and the removal or addition of cases/lots to a pallet or storage location;
   For the removal of a case or lot from the slot:

$${n}_{t} \to {n}_{t + 1} = {n}_{t} - p$$

$${C}_{i , t} \to {C}_{i , t + 1} = {C}_{i , t} - {S}_{i} ;$$

For the addition of a case or lot to the slot:

$${n}_{t} \to {n}_{t + 1} = {n}_{t} + q$$

$${C}_{i , t} \to {C}_{i , t + 1} = {C}_{i , t} + {Q}_{i} ;$$

Repeat the calculation of Tᵢ, Sᵢ, and Pᵢ for subsequent outgoing sales orders based on chronological order of transaction IDs and values at (t+1);
   This may be performed by writing the values of (n, Cᵢ) at (t+1) to the location where the values at (t) were accessed from previously.

As mentioned with regards to FIGS. 2 and 3, the data processing stages described with regards to FIG. 5 are based on the situation of a random selection of cases or lots from a storage location. If instead, a first-in-first-out (FIFO) selection process is used, then the stages would implement the corresponding operations or functions (i.e., those described with reference to FIG. 1(d)). Similarly, if a hybrid selection process is used, then then the modules would contain instructions for implementing the corresponding operations or functions (i.e., those described with reference to FIG. 1(e) and the associated formulas).

The application modules and/or sub-modules may include a suitable computer-executable code or set of instructions (e.g., as would be executed by a suitably programmed processor, microprocessor, or CPU), such as computer-executable code corresponding to a programming language. For example, programming language source code may be compiled into computer-executable code. Alternatively, or in addition, the programming language may be an interpreted programming language such as a scripting language.

Each application server (e.g., as represented by element 422 of FIG. 4) may include each application module. Alternatively, different application servers may include different sets of application modules. Such sets may be disjoint or overlapping.

The data storage layer 520 may include one or more data objects 522 each having one or more data object components 521, such as attributes and/or behaviors. For example, the data objects may correspond to tables of a relational database, and the data object components may correspond to columns or fields of such tables. Alternatively, or in addition, the data objects may correspond to data records having fields and associated services. Alternatively, or in addition, the data objects may correspond to persistent instances of programmatic data objects, such as structures and classes. Each data store in the data storage layer may include each data object. Alternatively, different data stores may include different sets of data objects. Such sets may be disjoint or overlapping.

Note that the example computing environments depicted in FIGS. 3, 4, and 5 are not intended to be limiting examples. Further environments in which an embodiment may be implemented in whole or in part include devices (including mobile devices), software applications, systems, apparatuses, networks, SaaS platforms, laaS (infrastructure-as-a-service) platforms, or other configurable components that may be used by multiple users for data entry, data processing, application execution, or data review.

The embodiments disclosed and/or described herein can be implemented in the form of control logic using computer software in a modular or integrated manner, Based on the disclosure and teachings provided herein, a person of ordinary skill in the art will know and appreciate other ways and/or methods to implement one or more embodiments using hardware and a combination of hardware and software.

In some embodiments, certain of the methods, models or functions described herein may be embodied in the form of a trained neural network or machine learning (ML) model, where the network or model is implemented by the execution of a set of computer-executable instructions. The set of instructions may be stored in (or on) a non-transitory computer-readable medium and executed by a programmed processor or processing element. The set of instructions may be conveyed to a user through a transfer of instructions or an application that executes a set of instructions (such as over a network, e.g., the Internet). The set of instructions or an application may be utilized by an end-user through access to a SaaS platform or a service provided through such a platform. The specific form of the method, model or function may be used to define one or more of the operations, functions, processes, or methods used in the development or operation of a neural network, the application of a machine learning technique or techniques, or the development or implementation of an appropriate decision process. Note that a neural network or deep learning model may be characterized in the form of a data structure in which are stored data representing a set of layers containing nodes, and connections between nodes in different layers are created (or formed) that operate on an input to provide a decision or value as an output.

In general terms, a neural network may be viewed as a system of interconnected artificial “neurons” that exchange messages between each other. The connections have numeric weights that are “tuned” during a training process, so that a properly trained network will respond correctly when presented with an image or pattern to recognize (for example). In this characterization, the network consists of multiple layers of feature-detecting “neurons”; each layer has neurons that respond to different combinations of inputs from the previous layers. Training of a network is performed using a “labeled” dataset of inputs in a wide assortment of representative input patterns that are associated with their intended output response. Training uses general-purpose methods to iteratively determine the weights for intermediate and final feature neurons. In terms of a computational model, each neuron calculates the dot product of inputs and weights, adds the bias, and applies a non-linear trigger or activation function (for example, using a sigmoid response function).

A machine learning model is a set of layers of connected neurons that operate to make a decision (such as a classification) regarding a sample of input data. A model is typically trained by inputting multiple examples of input data and an associated correct “response” or decision regarding each set of input data. Thus, each input data example is associated with a label or other indicator of the correct response that a properly trained model should generate. The examples and labels are input to the model for purposes of training the model. When trained (i.e., the weights connecting neurons have converged and become stable or within an acceptable amount of variation), the model will operate to respond to an input sample of data to generate the correct label or classification as an output.

In some examples, a neural network may be implemented based on multiple and/or different types of topologies and/or architectures including deep neural networks with fully connected (e.g., dense) layers, Long Short-Term Memory (LSTM) layers, convolutional layers, Temporal Convolutional Layers (TCL), other suitable types of deep neural network topology and/or architectures, or a combination thereof.

A neural network may have different types of output layers including, without limitation, output layers with logistic sigmoid activation functions, hyperbolic tangent activation functions, linear units, rectified linear units, other suitable types of nonlinear units, or a combination thereof.

With regards to the disclosed and/or described embodiments, once the supply chain starts supplying lot codes and sources more consistently, it should be feasible to collect data that can serve to validate and/or improve the performance of the disclosed approach and algorithm over time using ML. In particular, if an embodiment includes flexible parameters that are initially set, those could be “learned” over time for different products/distribution centers, or other factors.

Any of the software components, processes or functions described in this application may be implemented as software code to be executed by a processor using any suitable computer language such as Python, Java, JavaScript, C++, or Perl using conventional or object-oriented techniques. The software code may be stored as a series of instructions, or commands in (or on) a non-transitory computer-readable medium, such as a random-access memory (RAM), a read only memory (ROM), a magnetic medium such as a hard-drive, or an optical medium such as a CD-ROM.

In this context, a non-transitory computer-readable medium is almost any medium suitable for the storage of data or an instruction set aside from a transitory waveform. Any such computer readable medium may reside on or within a single computational apparatus and may be present on or within different computational apparatuses within a system or network. Further, the set of instructions may be conveyed to a user through a transfer of instructions or an application that executes a set of instructions (such as over a network, e.g., the Internet). The set of instructions or an application may be utilized by an end-user through access to a SaaS platform or a service provided through such a platform.

According to one example implementation, the term processing element or processor, as used herein, may be a central processing unit (CPU), or conceptualized as a CPU (such as a virtual machine). In this example implementation, the CPU or a device in which the CPU is incorporated may be coupled, connected, and/or in communication with one or more peripheral devices, such as display. In another example implementation, the processing element or processor may be incorporated into a mobile computing device, such as a smartphone or tablet computer.

The non-transitory computer-readable storage medium referred to herein may include a number of physical drive units, such as a redundant array of independent disks (RAID), a flash memory, a USB flash drive, an external hard disk drive, thumb drive, pen drive, key drive, a High-Density Digital Versatile Disc (HDDVD) optical disc drive, an internal hard disk drive, a Blu-Ray optical disc drive, or a Holographic Digital Data Storage (HDDS) optical disc drive, synchronous dynamic random access memory (SDRAM), or similar devices or other forms of memories based on similar technologies.

Such computer-readable storage media allow the processing element or processor to access computer-executable process steps and application programs stored on removable and non-removable memory media, to off-load data from a device, or to upload data to a device. As mentioned with regards to the embodiments disclosed and/or described herein, a non-transitory computer-readable medium may include almost any structure, technology, or method apart from a transitory waveform or similar medium.

Certain implementations of the disclosed technology are described herein with reference to block diagrams of systems, and/or to flowcharts or flow diagrams of functions, operations, processes, or methods. It will be understood that one or more blocks of the block diagrams, or one or more stages or steps of the flowcharts or flow diagrams, and combinations of blocks in the block diagrams and stages or steps of the flowcharts or flow diagrams, respectively, can be implemented by computer-executable program instructions. Note that in some embodiments, one or more of the blocks, or stages or steps may not necessarily need to be performed in the order presented or may not necessarily need to be performed at all.

These computer-executable program instructions may be loaded onto a general-purpose computer, a special purpose computer, a processor, or other programmable data processing apparatus to produce a specific example of a machine, such that the instructions that are executed by the computer, processor, or other programmable data processing apparatus create means for implementing one or more of the functions, operations, processes, or methods described herein. These computer program instructions may also be stored in a computer-readable memory that can direct a computer or other programmable data processing apparatus to function in a specific manner, such that the instructions stored in the computer-readable memory produce an article of manufacture including instruction means that implement one or more of the functions, operations, processes, or methods described herein.

While certain implementations of the disclosed technology have been described in connection with what is presently considered to be the most practical and various implementations, it is to be understood that the disclosed technology is not to be limited to the disclosed implementations. Instead, the disclosed implementations are intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, and to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural and/or functional elements that do not differ from the literal language of the claims, or if they include structural and/or functional elements with insubstantial differences from the literal language of the claims.

All references, including publications, patent applications, and patents, cited herein are hereby incorporated by reference to the same extent as if each reference were individually and specifically indicated to be incorporated by reference and/or were set forth in its entirety herein.

The use of the terms “a” and “an” and “the” and similar references in the specification and in the following claims are to be construed to cover both the singular and the plural, unless otherwise indicated herein or clearly contradicted by context. The terms “having,” “including,” “containing” and similar references in the specification and in the claims are to be construed as open-ended terms (e.g., meaning “including, but not limited to,”) unless otherwise noted.

Recitation of ranges of values herein are merely intended to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context.

The use of all examples, or exemplary language (e.g., “such as”) provided herein, is intended merely to better illuminate embodiments of the disclosure, and do not pose a limitation to the scope of the invention unless otherwise claimed. No language in the specification should be construed as indicating any non-claimed element as essential to each embodiment of the present invention.

As used herein in the specification, figures, and claims, the term “or” is used inclusively to refer items in the alternative and in combination.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the disclosure is not limited to the embodiments described or depicted in the drawings, and various embodiments and modifications can be made without departing from the scope of the claims below.

1. A method of identifying a source of a product believed responsible for an illness, contamination, or disease, comprising:
determining a number of cases (n) of a product that are presently stored in a location from which an order for the product will be fulfilled;
for each of one or more identifiers associated with a case, determining a number of the cases of the product presently stored in the location having each of a batch lot identifier (Cᵢ);
determining a number of cases to select from the location to fulfill a sales order (p);
based on the determined values, calculating the following values for the sales order:
batch lot proportion=Tᵢ=(Cᵢ/n), the proportion of the total cases in the sales order with a specified batch lot;
batch lot cases=Sᵢ=Tᵢ*p, the number of equivalent cases per batch lot;
batch lot probability=Pᵢ=1−((n−Cᵢ)/n)^{p}, the probability that a specific batch lot is selected for fulfilling the sales order;
storing data representing the values of Tᵢ, Sᵢ, and Pᵢ with data representing the sales order; and
providing the stored data and the data representing the sales order to an entity as part of complying with a regulation or otherwise assisting in tracking an illness, contamination, or disease to the use of a product from a specific production source.

2. The method of claim 1, wherein the product is a food product, or an ingredient used to prepare a food product.

3. The method of claim 1, further comprising updating one or more of the values of n or Cᵢ for the slot location as a result of the addition or removal of a case of the product from the location.

4. The method of claim 3, wherein the updating of one or more of the values of n or Cᵢ for the slot location further comprises:
representing a removal of one or more cases from the location by
$$cases available in slot = {n}_{t + 1} = {n}_{t} - p$$
$$cases per batch slot = {C}_{i , t + 1} = {C}_{i , t} - {S}_{i} ;$$
representing an addition of one or more cases to the location by
$$cases available in slot = {n}_{t + 1} = {n}_{t} + q$$
$$cases per batch slot = {C}_{i , t + 1} = {C}_{i , t} + {Q}_{i} ;$$
where, where q is the number of cases of the product added to the location and Qᵢ is the number of cases of the product added to the location and having a specific batch slot code.

5. The method of claim 1, wherein providing the stored data and the data representing the order to an entity as part of complying with a regulation or otherwise assisting in tracking a source of an illness, contamination, or disease arising from use of a product further comprises using the stored data and the data representing the order to determine which of multiple potential sources is the one most likely to be the source of the illness, contamination, or disease.

6. The method of claim 1, wherein the entity is a regulatory organization or an entity responsible for the production or distribution of the product.

7. The method of claim 1, further comprising one or more of:
altering a scope of a recall or investigation to expedite removal of implicated product and prevent additional consumer illness;
modifying one or more of warehouse and retail receiving, replenishment, and selection activities to optimize for traceability and product freshness;
optimizing supply chain sourcing to reduce mixed lot pallets; or
optimizing supply chain sourcing for product freshness, in response to the stored data and the data representing the sales order.
